(19) United States
(12) Patent Application Publication
(10) Pub. No.: US 2013/0281879 A1
(43) Pub. Date: Oct. 24, 2013

(54) DETERMINATION OF WHETHER A LUCIFERIAN CAN BE REHABILITATED

(71) Applicant: FIRST PRINCIPLES, INC., ALBANY, NY (US)

(72) Inventor: Keith A. Raniere, Clifton Park, NY (US)

(21) Appl. No.: 13/839,361

(22) Filed: Mar. 15, 2013

Related U.S. Application Data
(63) Continuation-in-part of application No. 12/272,941, filed on Nov. 18, 2008.
Application No. 12/272,941 is a continuation of application No. 11/931,524, filed on Oct. 31, 2007, now abandoned.

(51) Int. Cl. A61B 5/16

(52) U.S. Cl. CPC A61B 5/165
USPC 600/558; 600/300; 600/559

(57) ABSTRACT

An aspect of the present invention is a method for determining whether a Luciferian can be rehabilitated. The method includes stimulating the Luciferian with a first stimulus and recording a first physiological response by the Luciferian to said first stimulus. The method includes determining, from the first physiological response, a first polarity of the Luciferian's response to the first stimulus. The method includes stimulating the Luciferian with a second stimulus and recording a second physiological response by the Luciferian to said second stimulus. Further, the method includes determining, from the second physiological response, a second polarity of the Luciferian's response to the second stimulus. The method includes comparing the first polarity to the second polarity to determine whether the second polarity has a greater value than the first polarity; and determining, from said comparing of first polarity and second polarity, whether the Luciferian can be rehabilitated.

### RELATED APPLICATIONS

This application is a continuation in part of Ser. No. 12/272,941, filed on Nov. 18, 2008, which is a continuation of Ser. No. 11/931,524, filed on Oct. 31, 2007.

### BACKGROUND

1. Technical Field

The present invention relates to a method for determining whether a person, who has destructive tendencies, may be rehabilitated.

2. Related Art

Organizations such as businesses, religions, governments, law enforcement agencies, schools, etc. have had difficulties when individuals disrupt the organization's well-being by committing destructive acts. If organizations can identify such individuals they may better interact with them. Since these destructive individuals do not overtly disclose their nature, it is not easy to detect their presence. Failure to discover these individuals could be costly due to their destructive inclinations. There is a need to determine whether a given person, who has destructive tendencies, may be rehabilitated.

### SUMMARY

The present invention provides an objective and accurate capability for determining whether a Luciferian may be rehabilitated. A Luciferian is a person, who commits destructive acts. A Luciferian is a type of sociopath. He has severe behavioral problems that are masked by good psychological adjustment. He appears on the outside to be sane but he is not. A Luciferian is fundamentally unhappy and feels enmity toward others but has learned that, for him, pleasure can be had from achieving his selfish desires such as destroying material objects or relationships, and causing others pain, distress, or unhappiness. A Luciferian realizes his desires by any means—without consideration for others and without remorse. A Luciferian is a person lacking in conscience and loyalty to others. He commonly employs manipulation and deceit to achieve his desired end and is therefore capable of acts that could be highly destructive to those that interact with him. A Luciferian, therefore, typically experiences pleasure or gratification in situations where “normal” people would be repulsed or disturbed.

A first general aspect of the present invention is a method for determining whether a Luciferian can be rehabilitated, comprising: stimulating said Luciferian with a first stimulus; recording a first physiological response by said Luciferian to said first stimulus; determining, from said first physiological response, a first polarity of said Luciferian's physiological response to said first stimulus; stimulating the Luciferian with a second stimulus; recording a second physiological response by the Luciferian to said second stimulus; determining, from said second physiological response, a second polarity of the Luciferian's physiological response to said second stimulus; comparing said first polarity to said second polarity to determine whether said second polarity has a greater value than said first polarity; and determining, from said comparing of said first polarity and said second polarity, whether said Luciferian can be rehabilitated.

A second general aspect of the present invention is a method for determining a humanity of a Luciferian, comprising: stimulating said Luciferian with a plurality of constructive stimuli; recording a physiological response by said Luciferian corresponding to each constructive stimulus of said plurality of constructive stimuli; determining, for said each constructive stimulus, a corresponding polarity of said physiological response; comparing said corresponding polarity of said physiological response for said each constructive stimulus to determine a greatest value; and determining, from said greatest value, said humanity of said Luciferian.

A third general aspect of the present invention is a method for determining a humanity of a Luciferian, comprising: stimulating said Luciferian with a plurality of destructive stimuli; recording a physiological response by said Luciferian corresponding to each destructive stimulus of said plurality of destructive stimuli; determining, for said each destructive stimulus, a corresponding polarity of said physiological response; comparing said corresponding polarity of said physiological response for each destructive stimulus to determine a least value; and determining, from said least value, said humanity of said Luciferian.

### BRIEF DESCRIPTION OF THE DRAWINGS

FIG. 1 depicts a representation of a human brain identifying the locations of the ventral tegmental area, the nucleus accumbens, and the prefrontal cortex.

FIG. 2 depicts a person's relative change in dopamine levels as represented by positron emission tomography (PET) scans.

FIG. 3 depicts a person's physiological responses to a stimulus, said physiological responses being recorded by instrumentation, said physiological responses being processed according to an algorithm to determine whether the person is a Luciferian.

FIG. 4 depicts a space of potential polarities in relation to the stimulus of FIG. 3 and a subspace of the space, wherein the subspace defines a Luciferian.

FIG. 5 depicts an event stream comprised by the stimulus of FIG. 3.

FIG. 6 represents the stimulus of FIG. 3 as having N stimulus components with an associated N polarity components, wherein N∃2.

FIG. 7 represents the N stimulus components and N polarity components of FIG. 6 as an N-component stimulus vector S and an associated N-component polarity vector P.

FIG. 8 depicts a flow chart for determining whether the person in FIG. 3 is a Luciferian.

FIG. 9 depicts the flow chart of FIG. 8 with added steps relating to the space of FIG. 4.

FIG. 10 depicts a person's response to items on a questionnaire, said responses being recorded by instrumentation, said responses being processed according to an algorithm to determine whether the person is a Luciferian.

FIG. 11 depicts a flow chart for determining whether the person in FIG. 10 is a Luciferian.

FIG. 12 depicts the flow chart of FIG. 11 with added steps relating to the space of FIG. 4.

FIG. 13 depicts a flow chart for generating a polarity profile of a questionnaire.

FIG. 14 depicts a flow chart for determining whether the Luciferian may be rehabilitated.

FIG. 15 depicts a flow chart for determining a humanity of a Luciferian.

FIG. 16 depicts a flow chart for an alternative method of determining a humanity of a Luciferian.

FIG. 17 depicts the recording and processing a response to a stimulus.

### DETAILED DESCRIPTION

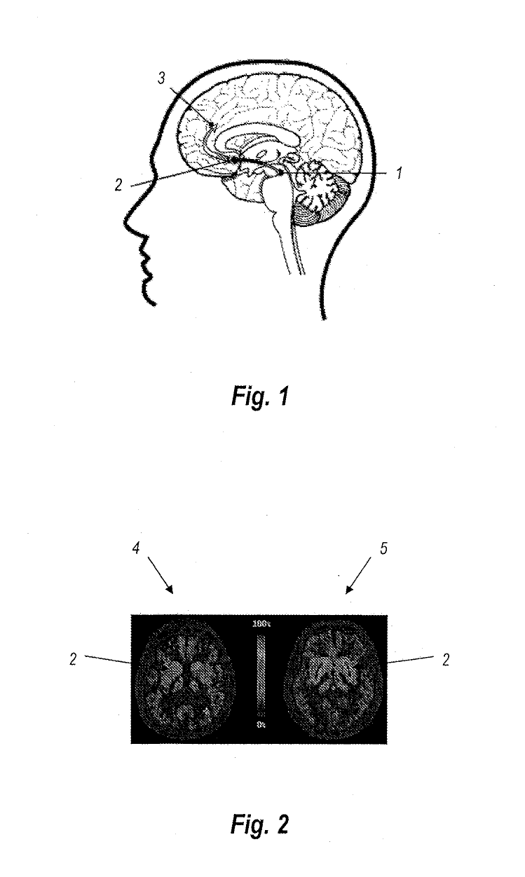

Strong feelings of satisfaction are associated with behaviors that meet various human needs. For example, specific areas in the human brain provide pleasurable sensations as a “reward” for these behaviors. These areas are interconnected with one another to form what is known as the “reward circuit”. The reward circuit in the brain provides feelings of satisfaction to encourage us to repeat pleasurable experiences that we learn in the course of our lives. The reward circuit is at the heart of our mental activity and guides all our behaviors. This circuit is complex, but it contains a central link that plays a fundamental role. This link is comprised of the nerve connections between two particular small groups of neurons. FIG. 1 contains a depiction of the human brain and the location of these groups of neurons. One of these groups is located in the ventral tegmental area (VTA) 1, and the other in the nucleus accumbens 2. The chemical messenger that makes the connections between these two groups of neurons is dopamine. Dopamine is a chemical messenger (neurotransmitter) in the brain. Thus, there is an association between feelings of pleasure and dopamine. For example, when the cortex has received and processed a sensory stimulus indicating a reward, it sends a signal announcing this reward to the VTA 1 whose activity then increases. The VTA 1 then releases dopamine, not only into the nucleus accumbens 2 but also into the septum, the amygdala, and the prefrontal cortex 3. The nucleus accumbens 2 then activates the individual's motor functions, while the prefrontal cortex 3 focuses his or her attention. These regions are part of the reward circuit. The reward circuit is part of the medial forebrain bundle (MFB), whose activation leads to the repetition of the gratifying action to strengthen the associated pathways in the brain.

The MFB is a bundle of axons that originates in the reticular formation, crosses the VTA, passes through the lateral hypothalamus, and continues into the nucleus accumbens as well as the amygdala, the septum, and the prefrontal cortex. The MFB is composed of ascending and descending pathways, including most of the pathways that use monoamines as a neurotransmitter. The mesocorticolimbic dopaminergic system is one of its main components. This system comes into play when a person experiences pleasure.

Another distinct pleasure circuit, driven by opioids, overlaps considerably with the dopamine system, to the point where some cells take part in both circuits. A key part of this system is the ventral pallidum which takes in signals from the nucleus accumbens and passes them on to the cortex. Additionally, cells nearer the surface of the brain, in a region of the forebrain called the orbitofrontal cortex (OFC), may have a vital role as well.

Pleasure is a direct response to a sensory stimulus. One of the first brain regions to process sensory information is the OFC. Signals such as taste, touch, smells, and sounds travel first to the sensory cortex, and from there go straight to the OFC. Visual information enters by a slightly less direct route. From here the signal passes into the opioid and dopamine circuits. A pleasant or unpleasant smell, taste, or touch is represented in the sensory cortex simply as a magnitude—strong brain activity for a strong smell and weak brain activity for a faint one, for example. But by the time it has passed on to the OFC it now correlates with how pleasant or unpleasant the stimulus may be rated. As each sensory signal passes through the OFC, it may receive a level of liking or disliking Activity in the OFC may increase or decrease in line with the level of pleasure people may feel for a given stimulus.

The ventral pallidum and OFC may be associated with pleasure, but other parts of the brain's cortex may also become especially active during pleasure such as the cingulate cortex and somatosensory cortex. The emotion of pleasure may include the dopamine and opiate circuits and OFC, but actually feeling pleasure may require a level of reflection that can only be provided by other brain regions that carry sensory maps or representations of our bodies—the cingulate cortex, somatosensory cortex and certain parts of the brainstem.

Testing for physiological evidence of elevated activity in the reward or pleasure center of the brain has not been used to determine whether a Luciferian may be rehabilitated or to determine a Luciferian's humanity. The present invention seeks to determine whether a Luciferian takes more pleasure in some aspect of human welfare, animal welfare, human value or human dignity than the pleasure that the Luciferian takes in destruction.

Ser. No. 12/272,941 provides testing for physiological evidence of elevated activity in the reward or pleasure center of the brain used as a way to ascertain whether a person is a Luciferian. Physiological characteristics which can be independent, dependent, or interdependent with the pleasure center can be used as indicators of whether a person is a Luciferian such as changes in heart rate, blood pressure, respiration depth, electrodermal response, skin temperature, sweating, muscle electrical activity, and cerebral blood flow.

Testing to determine whether a person is a Luciferian may include subjecting a person to a stimulus. A response to a stimulus may be an emotional, chemical, or physiological response which may be expressed as a polarity (P). The polarity of the response of a person to a stimulus is indicative of an extent to which the emotional, chemical, or physiological response is positive or negative. For example, a scale of polarity may be defined such that: P=0 is a neutral point at which the response is neither positive nor negative; P>0 may denote a positive response such that P increases as the magnitude of the positive response increases; and P<0 may denote a negative response such that P algebraically decreases as the magnitude of the negative response increases.

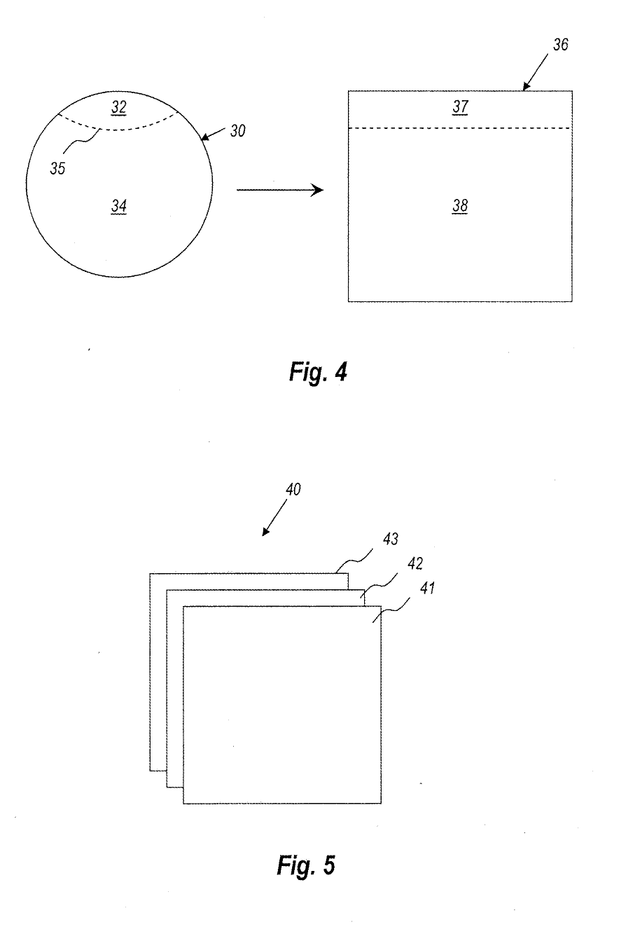

In FIG. 4, the space 30 is the locus of all potential emotional, chemical, and physiological polarities of the polarity of the response of the person 10 to the stimulus S. In other words, the space 30 includes all possible polarities that could manifest by any arbitrary person subjected to the stimulus S. FIG. 4 also shows the space 30 as consisting of a subspace 32 and a subspace 34. If the algorithm 22 determines that the polarity of the person 10 falls within the subspace 32 then the person 10 is determined to be a Luciferian. Conversely, if the algorithm 22 determines that the polarity of the person 10 falls within the subspace 34 or on the interfacial boundary 35 between subspaces 32 and 34, then the person 10 is determined not to be a Luciferian. Such determination of which subspace (32 or 34) the polarity of the person 10 falls within could also be made by a human being instead of by the algorithm 22 within the computer 24, wherein the human being would utilize the same methodology as is utilized by the algorithm 22.

The space 30, as well as the subspaces 32 and 34 individually, could be represented in any electronic dataset format as may exist in, inter alia, a database, table, file, spreadsheet, etc. FIG. 4 shows such a dataset 36 for representing the space 30, wherein a portion 37 of the dataset 36 represents the subspace 32, and wherein a portion 38 of the dataset 36 represents the subspace 34. Alternatively, the portion 38 may not be present if the portion 37 is present, since the algorithm 22 need only test for whether the polarity of the person 10 falls within the subspace 32; thus the algorithm 22 needs to know the scope of the subspace 32 and does not need to know the scope of the subspace 34. Similarly, the portion 37 may not be present if the portion 38 is present, since the algorithm 22 need only test for whether the polarity of the person 10 does not fall within the subspace 34; thus the algorithm 22 needs to know the scope of the subspace 34 and does not need to know the scope of the subspace 32.

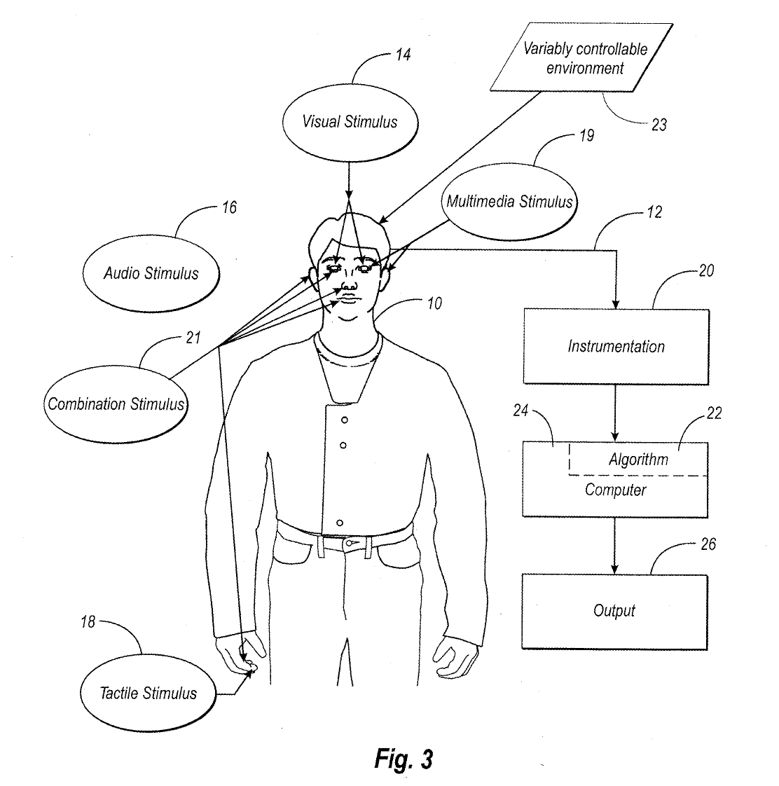

FIG. 3 depicts a recording and processing of responses 12 of a person 10 to a stimulus (S). The depiction in FIG. 3 may be referred to as a test to determine whether a person is a Luciferian. A test to determine whether a person is a Luciferian comprises a person subjected to a stimulus and a recording of a response to the stimulus. The stimulus S is a factor capable of eliciting a response from a person. The stimulus S may be a visual stimulus 14, an audio stimulus 16, a tactile stimulus 18, a multimedia stimulus 19, or any other stimulus. A combination stimulus 21 may be any combination of a visual stimulus 14, an audio stimulus 16, a tactile stimulus 18, a multimedia stimulus 19, or any other stimulus. The stimulus S may be obvious or unobvious as a test element to the person 10 subjected to a test. The person 10 may be in a variably controllable environment 23 while subjected to the stimulus S. Controllable environmental conditions may include temperature, humidity, air movement, lighting, room size, room shape, location, setting, odor, noise, or any other external physical condition surrounding the person 10. A response to a stimulus may include physiological, emotional, cognitive, oral, written, or other like responses by a person to a stimulus. Physiological responses 12 may be recorded by instrumentation 20 and subsequently processed according to an algorithm 22 stored in a data processing device 24 to determine, and to display as output 26, whether the person 10 is a Luciferian.

The stimulus S may be of a type that evokes a response from the person 10. The stimulus S may include one of, or a combination of, the visual stimulus 14, the audio stimulus 16, the tactile stimulus 18, the multimedia stimulus 19, or any other factor or combination of factors capable of eliciting a response from the person 10. Examples of the visual stimulus 14 include, inter alia, a movie; a series of pictures; a sports telecast, a sequence of colors; etc. Examples of the audio stimulus 16 include, inter alia, a recital of a book, emotionally provocative sounds; expressions of anger, fear, pain, laughter; white noise, subliminal signals; etc. White noise is a random signal with a flat power spectral density. In other words, the signal's power spectral density has equal power in any band, at any center frequency, having a given bandwidth. Examples of the tactile stimulus 18 include, inter alia, touching a dead person or dead animal. As a response to tactile stimulus of touching the dead person or dead animal, if the individual is Luciferian, one response in the subset would be a positive polarity based on the increase of chemical and electrical activity in the “reward circuit”.

The multimedia stimulus 19 may include combinations of the visual and audio stimuli such as combinations of text, sound, graphics, animation, photo images, and full-motion video. A combination stimulus 21 may include any combination of visual, audio, tactile, multimedia, or any other stimulus. An example of an audiovisual stimulus that would increases the response of the of the reward circuit for a Luciferian would be a video depicting the failure of an athlete by falling just before the finish line and losing or a skier crashing during a great athletic event. This would result in a positive polarity for the Luciferian. A negative polarity would be if the Luciferian were to see the athlete or skier be successful.

The instrumentation 20 serves to record the physiological responses 12, which may include involuntary or unintentional responses, under the assumption that the physiological responses 12 could be used to infer the responses of the person 10 to the stimulus S. Medical instrumentation 20 may be devices which are used to measure attributes of human physiology. The variable measured may include any measurable variable related to human physiology. Examples of instrumentation 20 may include, inter alia, medical instrumentation such as an electroencephalogram (EEG) instrument, a computed axial tomography (CAT) instrument, a positron emission tomography (PET) instrument, a magnetic resonance imaging (MRI) instrument, an electromyogram (EMG) instrument, a polygraph instrument, or combinations thereof.

For example, FIG. 2 contains PET scan images evidencing increased levels of dopamine in the brain—particularly in the nucleus accumbens 2. PET scanners may be used to view brain activity in test subjects who have been injected with a chemical that binds to dopamine receptors in the brain, but is less able to bind when the brain is releasing dopamine. A decrease in binding to the receptors is associated with an increase in dopamine release, while an increase in binding indicates reduced release of dopamine. This technique provides a visualization of the strength and location of dopamine release. The PET scan images in FIG. 2 illustrate an increase of dopamine 4 relative to a baseline level 5. Scans show the concentration of available dopamine receptors on a scale that goes from blue (low) to red (high). More dopamine receptors available means less dopamine is in the brain. PET scans may be used in this way to indicate when a test subject experiences an increase of dopamine in response to a stimulus.

Examples of a polarity of P=0 would be for a person's normal chemical balance of dopamine or normal neural activity, based on statistical sampling and given that the individual does not have drugs in his system, drug addiction, mental or physical illness, obesity, or other factors that may skew normal dopamine or neural activity levels.

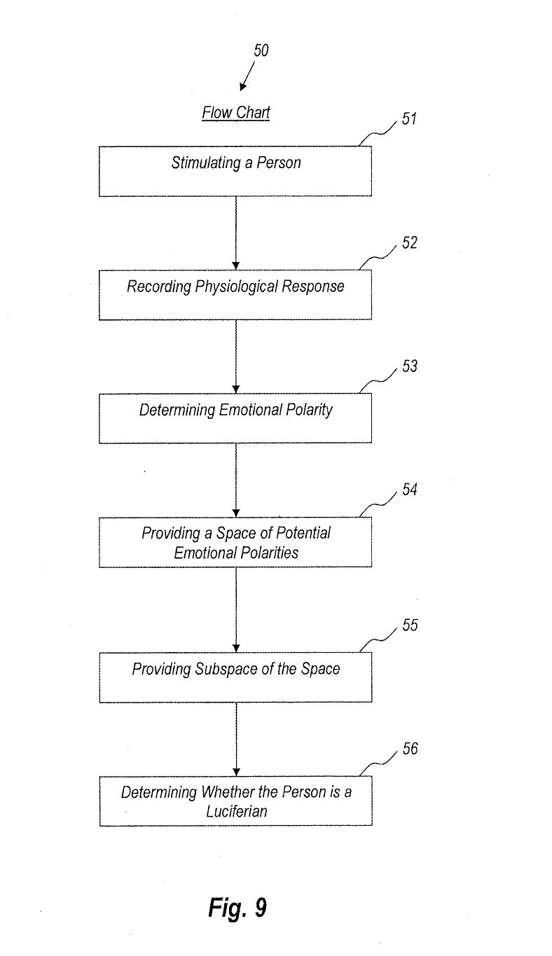

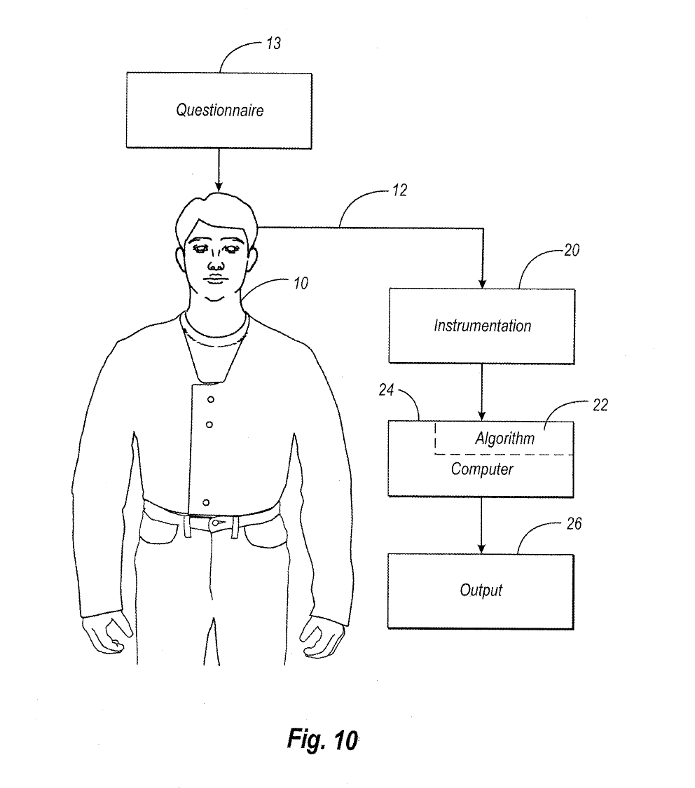

The responses 12 may include voluntary or intentional responses such as responding to items on a questionnaire as will be discussed infra in conjunction with FIGS. 9 and 10 or the responses 12 may include involuntary or unintentional physiological responses such as, inter alia, changes in heart rate, blood pressure, respiration depth, electrodermal response, skin temperature, muscle electrical activity, cerebral blood flow, and neural activity in pleasure centers of the brain. If the responses 12 include such voluntary or intentional physiological responses, then the instrumentation 20, such as a polygraph instrument, may be used to ascertain whether the person 10 has been truthful in his or her voluntary or intentional responses.

Polarity may be inferred from data collected by medical instrumentation 20 such as EEG, CAT, PET, MRI, EMG, and polygraph. For example, EEG may be used to assess brain electrical activity. While connected to EEG instrumentation, a test subject may be subjected to a stimulus S. Electrical signals produced by the brain neurons in response to the stimulus S are picked up by electrodes and may be transmitted to a polygraph for analysis. Similarly, PET and CAT scans may be used to obtain information about brain function and to study brain activity in response to a stimulus S. EEG, PET, and CAT recordings may be analyzed for abnormalities to reveal a polarity to a given stimulus S. The recorded physiological responses 12 of the person 10 may be processed by an algorithm 22 executed by a computer 24. Generally, any data processing hardware (e.g., a microprocessor) capable of executing compiled code, interpreted code, or hard-wired code could be used instead of the computer 24. Alternatively, the recorded physiological responses 12 could be processed by a human being instead of by data processing hardware. The output 26 expresses the result of the processing of the recorded physiological responses 12, and said output 26 may include an expression of whether or not the person 10 is a Luciferian.

The algorithm 22 determines the polarity of the response of the person 10 to the stimulus S, and further determines whether the person is a Luciferian. After the algorithm 22 determines said polarity, the algorithm 22 may utilize a space 30, as exemplified in FIG. 4 to determine whether the person is a Luciferian.

The algorithm 22 may be executed in real time in direct response to the recording of the physiological responses 12 by the instrumentation 20. For said real-time execution of the algorithm 22, the algorithm 22 should be electronically coupled to the physiological data recorded by the instrumentation 20. Alternatively, the algorithm 22 may be executed off-line after the recording of the physiological responses 12 by the instrumentation 20. For said off-line execution of the algorithm 22, the algorithm 22 may or may not be electronically coupled to the physiological data recorded by the instrumentation 20.

Using well-known concepts of physiology and medical instrumentation, the subspace 32 within the space 30 may be generated. A test to determine whether a person is a Luciferian comprises a person subjected to a stimulus S and recording of a response to the stimulus S, such as that depicted in FIG. 3. A test to determine whether a person is a Luciferian may be designed to identify the types of stimuli that elicit pleasure responses in a test subject. For example, a person may be subjected to a neuropsychological test comprising a stimulus S. Neuroimaging, using medical instrumentation 20 may be used to identify and/or quantify neurophysiological processes 12 in the pleasure systems of the brain in response to the neuropsychological test stimulus S. Based on the stimulus S, and the corresponding response 12, a polarity may be determined. A space 30 may be created for each stimulus S. Luciferian-type polarities are a sub space 32 of the space 30. The sub space 32 may be identified directly by defining the range of polarities a Luciferian would produce or the sub space 32 may be identified indirectly by defining what range of polarities a non-Luciferian would produce within the space 30, and selecting all others as the sub space 32.

One example of a test to determine whether a person is a Luciferian comprises a video clip of an ice skater performing in a competition. Everything goes flawlessly until, at the very end, a mistake is made during a jump and the skater falls to the ice—all hope of winning the event is gone. A Luciferian will experience pleasure and satisfaction from this. Accordingly, the Luciferian's neurophysiological processes 12 will register pleasure over the skater's misfortune which may be identified and quantified using the medical instrumentation 20. On the other hand, a non-Luciferian will feel sympathetic and empathize with the unfortunate skater. Unlike the Luciferian, a non-Luciferian's neurophysiological processes 12 will not register as pleasure for this stimulus.

Another example of a test to determine whether a person is a Luciferian that may be given is to have the subject view a film of human torture. Instrumentation 20 will reveal that a Luciferian subject viewing a torture movie is experiencing a pleasure response. Further, external stimuli (i.e. not included in the torture movie) such as a door slamming will not elicit much response, if any, from a Luciferian subject. A Luciferian may be enjoying the scene too much to divert attention to other things. This may also be due to a low startle response typical of sociopaths. In contrast, when a non-Luciferian views a torture movie, pleasure centers in the brain are not responsive and may even diminish from a previous level. A non-Luciferian will exhibit a greater response to external stimuli such as a door slamming. This may be due to a desire to direct attention elsewhere rather than on the uncomfortable scene in the movie. The preceding examples are merely illustrative and any other criteria for defining the scope of a Luciferian.

A test to determine whether a person is a Luciferian may comprise elements designed to identify a person's true physiological response to stimuli. A Luciferian may fear discovery and may attempt to outwit a test that would reveal his true character. A Luciferian may try to control his responses, possibly by disinterest or unresponsiveness to certain types of stimuli or possibly by mimicking responses he feels would be appropriate from “normal” people. Accordingly, a test may have obvious stimulus S test elements (e.g. questionnaire, movie clips, pictures, etc.), but a test may also incorporate stimulus S test elements which are unobvious. A test may contain stimuli that a subject does not realize are part of the test. There may be unobvious test stimuli that are being administered before, during, or after, the obvious test elements. Unobvious test stimuli may be of any type including visual 14, audio 16, tactile 18, multimedia 19, and combination 21. For example, unobvious test stimuli may be subliminal audio or video signals, a slamming door, a person tripping on a sidewalk while visible to a subject through an outside window, or any other stimuli that may be administered to a subject without the subject's recognition that the stimuli is part of a test. Unobvious stimuli may be the primary test or a part of the primary test. On the other hand, responses to unobvious test stimuli may confirm or bring into question the responses to the obvious test stimuli. In this way, true responses may be captured from a Luciferian attempting to “beat” a test.

The stimulus S of FIG. 3 may be an event stream such as the event stream 40 shown in FIG. 5, in accordance with embodiments of the present invention. In FIG. 5, the event stream 40 includes events 41, 42, and 43. Although FIG. 5 shows 3 events, the number of such events may be any positive integer of at least 1. An example of the event stream 40 is a sequence of events occurring in an activity such as, inter alia, a movie in which the events 41, 42, and 43 each represent a scene or scenery occurring in a time interval within the movie. The event stream 40 may include the entire activity (e.g., the movie), or the event stream 40 may include a continuous or discontinuous subset of the activity, wherein the subset is not the entire activity. Other examples of event streams include a movie, a play, a television performance, a sports event, and a military event. The event stream 40 may be presented in a real-time mode; e.g., if the event is a sports event such as a football game then the football game would presented as the stimulus S to the person 10 in real time while the football game is being actually played. The event stream 40 may alternatively be presented in a playback mode; e.g., if the event is a sports event such as a football game then the football game would first be recorded (such as on video tape) and subsequently presented (by being played back) as the stimulus S to the person 10.

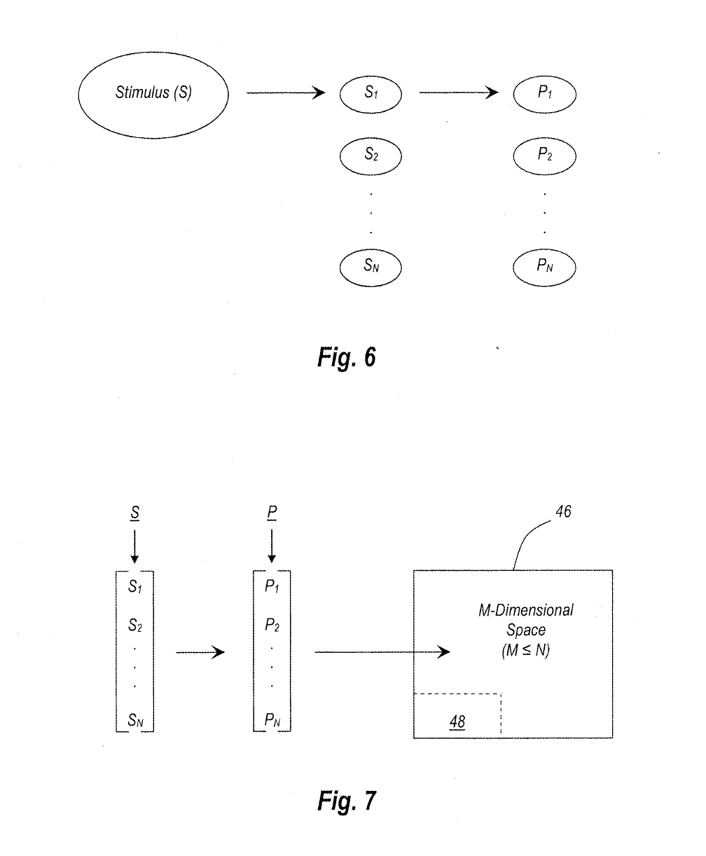

If the stimulus S of FIG. 3 comprises N events (N∃1) of an event stream, then the stimulus S may comprise N components S₁, S₂, . . . , S_{N} respectively corresponding to the N events of the event stream. For each such stimulus component Sᵢ (i=1, 2, . . . , N), there is an associated polarity Pᵢ of the response of the person 10 to the stimulus component Sᵢ. Thus, the N polarities P₁, P₂, . . . , P_{N} are respectively associated with the N stimulus components S₁, S₂, . . . , S_{N} as depicted in FIG. 6, in accordance with embodiments of the present invention.

S is defined herein as an N-component stimulus vector having components S₁, S₂, . . . , S_{N} as depicted in FIG. 7, in accordance with embodiments of the present invention. FIG. 7 also shows that the stimulus vector S results, via the configuration of FIG. 3, in an N-component polarity vector P having components P₁, P₂, . . . , P_{N}. In FIG. 7, an M-dimensional space 46 embodies the space 30 of FIG. 4, and the subset 48 of the space 46 embodies the subset 32 of the space 30 of FIG. 4. If P₁, P₂, . . . , P_{N} are mutually independent, then M=N. If one of P₁, P₂, . . . , P_{N} is dependent upon another of P₁, P₂, . . . , P_{N}, then M<N, wherein N−M is the number of such mutual dependencies. FIG. 7 shows a projection 45 of the polarity vector P into the space 46 to determine if the polarity vector P is within the subspace 48. If the polarity vector P is determined, such as by the algorithm 22 of FIG. 3, to be within the subspace 48, then the person 10 of FIG. 3 has is determined to be a Luciferian.

A special case of FIG. 7 arises for N=1 in which the stimulus S has exactly one stimulus component. In this special case N=M=1 and there is one response and one associated polarity. For this special case, the space 30 of FIG. 4, or the space 46 of FIG. 7, thus comprises a one-dimensional range of polarities.

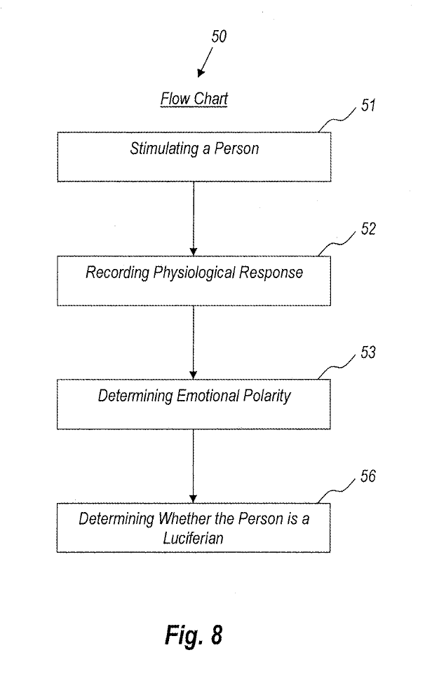

FIG. 8 depicts a flow chart 50 that describes steps in a method for determining whether the person 10 in FIG. 3 is a Luciferian. The flow chart 50 presents an alternative view in conjunction with FIGS. 3-7. The flow chart 50 comprises steps 51, 52, 53, and 56. Referring to both FIG. 8 and FIG. 3, the step 51 stimulates the person 10 with the stimulus S. The step 52 records the response 12 by the person 10 to stimulus S. The step 53 determines, from the response 12, a polarity of the response of the person 10 to the stimulus S. The step 56 determines, from said polarity of the response of the person 10 to the stimulus S, whether the person 10 is a Luciferian.

FIG. 9 depicts the flow chart of FIG. 8 with added steps 54 and 55 relating to a space such as the space 30 of FIG. 4 or the space 46 of FIG. 7. The step 54 provides the space. The step 55 provides a subspace of the space associated with step 54 such that if the polarity of the response of the person 10 to the stimulus S is within said subspace, then the person is determined to be a Luciferian.

FIG. 3 may be modified to reflect an alternative mechanism for determining whether the person 10 is a Luciferian, using a “questionnaire” as the stimulus S instead of or in addition to, for example, the visual stimulus 14, the audio stimulus 16, the tactile stimulus 18, the multimedia stimulus 19, and the combination stimulus 21. A questionnaire may be combined with other stimuli by containing items or questions related to the other stimuli. For example, a person may be shown a video followed by a questionnaire asking specific questions about the video. A questionnaire may be used in variably controllable environmental conditions 23. For example, a person may complete the same questionnaire two times—first at 70° F. and then at 85° F. Following this, the two sets of answers provided in response to the questionnaire may be compared for inconsistencies that may reveal a polarity of the person. Accordingly, FIG. 10 depicts a recording and processing of responses 12 of a person 10 to a stimulus S in the form of a questionnaire 13, in accordance with embodiments of the present invention. The questionnaire 13 may be administered to a person 10 in writing or orally. The responses 12 may represent voluntary or intentional responses by the person 10 to items on the questionnaire 13 and/or they may represent involuntary or unintentional responses. As in FIG. 3, the responses 12 may be recorded by instrumentation 20 and subsequently processed according to an algorithm 22 stored in a data processing device 24 to determine, and to display as output 26, whether the person 10 is a Luciferian. The instrumentation 20 may include any device (e.g., a sheet of paper, a computer diskette, etc.) that records physiological responses 12. The instrumentation 20 may include a polygraph instrument which may be used to ascertain whether the person 10 has been truthful in his or her voluntary or intentional responses, by correlating the voluntary response with the polygraph data.

Some or all of the responses by the person 10 may reflect a polarity of the person 10. Alternatively, at least one of the responses by the person 10 may reflect a polarity of the person 10, in order to make it difficult for the person 10 to guess, or otherwise try to determine, the purpose of each item on the questionnaire 13.

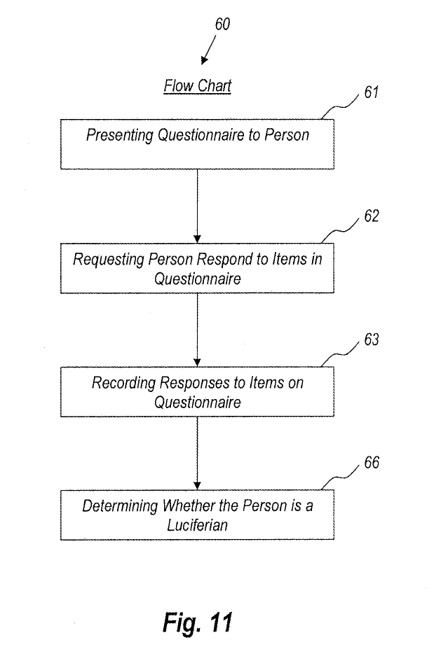

FIG. 11 depicts a flow chart 60 that describes steps in a method for determining whether the person 10 in FIG. 3 is a Luciferian. The flow chart 60 presents the embodiments associated with use of the questionnaire 13 of FIG. 10. The flow chart 60 comprises steps 61, 62, 63, and 66. Referring to both FIG. 11 and FIG. 3, the step 61 presenting the questionnaire 13 to the person 10, wherein the questionnaire 13 comprises a plurality of items. The step 62 requests the person 10 to communicate a voluntary response to each item of the plurality of items. The step 63 records the voluntary response of the person 10 to each item of the plurality of items. The step 66 determines, from said voluntary response of the person 10 to the items on the questionnaire 13, whether the person 10 is a Luciferian, based on a predetermined prescription. The algorithm 22 of FIG. 10 may include said predetermined prescription. Use of predetermined prescription is analogous to the space 30 and subspace 32 of FIG. 4 (or the space 46 and subspace 48 of FIG. 7), described supra.

FIG. 10, depicts the flow chart of FIG. 11 with added steps 64 and 65. The step 64 determining a polarity of the voluntary or intentional responses of the person 10 to each item of the plurality of items. The step 65 determines, from the polarity of the voluntary or intentional responses of the person 10 to each item of the plurality of items, whether the person is a Luciferian.

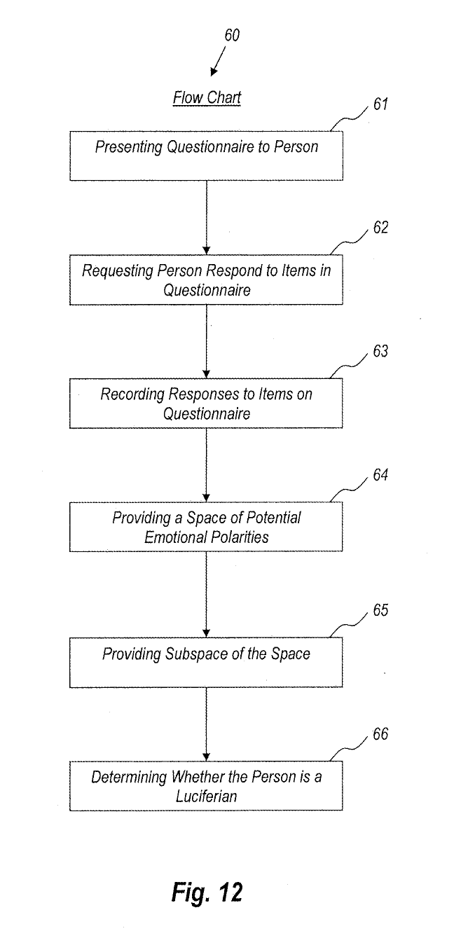

In FIGS. 11 and 12, the requesting step 62 may comprise requesting the person 10 to communicate a structured voluntary response to at least one item of the plurality of items. Said structured voluntary response may be selected by the person 10 from a predetermined set of responses for each item on the questionnaire 13.

Alternatively, the requesting step 62 may comprise requesting the person 10 to communicate an unstructured voluntary response to at least one item of the plurality of items. The predetermined prescription may include a comparison of the unstructured voluntary response with a predetermined set of responses.

In addition to recording the voluntary or intentional responses of the person 10, the instrumentation 20 may include instrument(s) that record involuntary or unintentional physiological responses by the person 10 while the person 10 is communicating voluntary or intentional responses of the person 10 to the items on the questionnaire 13.

All aspects of FIGS. 3-7, discussed supra, apply to use of the questionnaire 13 in FIG. 10 in conjunction with the flow chart 60 of FIGS. 11 and 12. Such aspects of FIGS. 3-9 include, inter alia, use of: the instrumentation 20, algorithm 22, computer 24, and output 26 of FIG. 3; the space 30 and dataset 36 of FIG. 4; the event stream 40 of FIG. 5; the stimulus components and associated vector S, the polarity components and associated vector P, and space 46 of FIGS. 6 and 7.

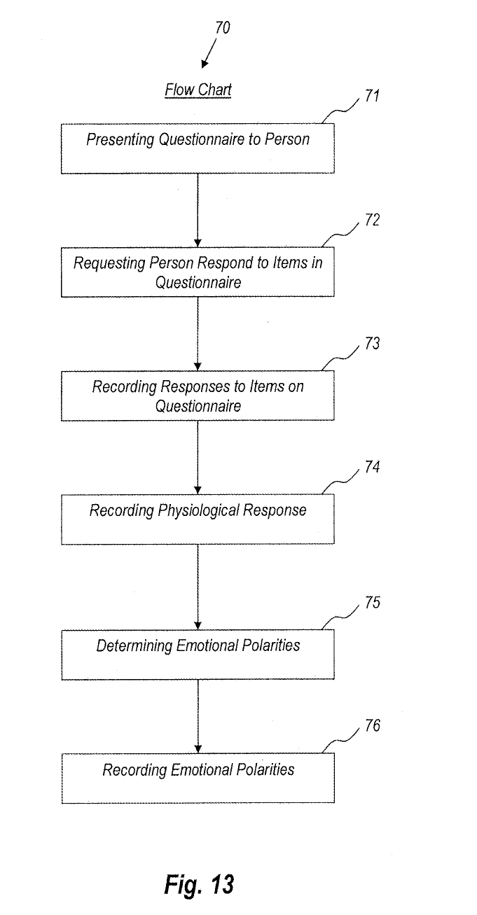

A method for generating a polarity profile of the questionnaire 13, comprises correlating recorded polarities with recorded voluntary responses in relation to the questionnaire 13. The recorded voluntary responses and the recorded polarities are derived from the following procedure, depicted in the flow chart 70 in FIG. 13, as applied to a symbolic person representing each individual of N individuals such that N is large enough for said correlating to be statistically significant. In FIG. 13, step 71 presents to the person the questionnaire 13 comprising a plurality of items. The step 72 requests the person to communicate a voluntary response to each item of the plurality of items. The step 73 records the voluntary responses of the person. The step 74 records physiological responses of the person while the person is communicating the voluntary responses. The step 75 determines, from the physiological responses, polarities of the voluntary responses. The step 76 records the polarities. The recorded voluntary responses of the person and recorded polarities are then utilized for performing said correlating.

A subjective evaluation may be used to supplement an objective evaluation of a person in the determination of whether the person is a Luciferian. For example, a person may observe a test subject during an objective test that includes stimuli and recording instrumentation, for signs that the test subject's responses were not genuine. Similarly, a person may look for signs of disingenuous responses during an interview with a test subject conducted independently or in conjunction with an orally administered questionnaire. Incorporating a subjective evaluation of a test subject may aid in the determination of whether the subject is a Luciferian.

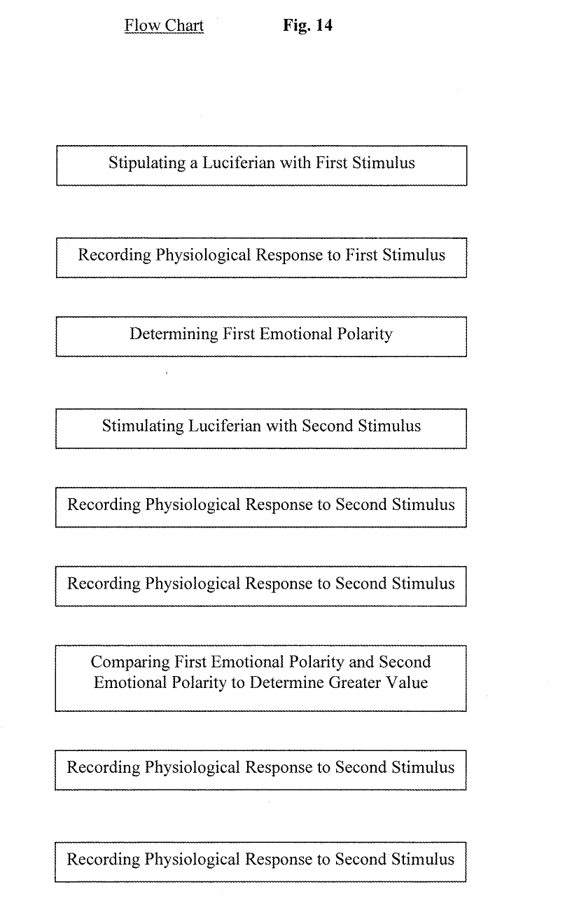

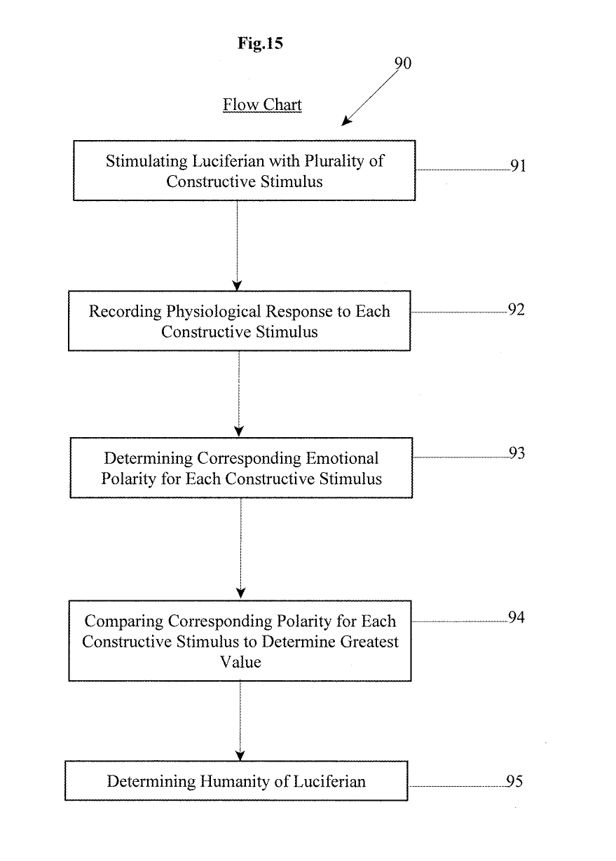

In accordance with FIG. 4, a person 10 is determined to be a Luciferian if the polarity of the person 10 falls within subspace 32. After determining that a person 10 is a Luciferian, it is helpful to determine whether the person 10 may be rehabilitated. FIG. 14 depicts a flow chart 80 for determining whether the person 10, pre-determined to be a Luciferian, may be rehabilitated, in accordance with the present invention. The flow chart 80 of FIG. 14 comprises the steps 81, 82, 83, 84, 85, 86, 87, and 88. Referring to FIG. 14, the step 81 stimulates the person 10 with a first stimulus. The step 82 records the response 12a by the person 10 to first stimulus. The step 83 determines, from response 12a, a first polarity of the response 12a of the person 10 to the first stimulus. The step 84 stimulates the person 10 with a second stimulus. The step 85 records the response 12b by the person 10 to second stimulus. The step 86 determines, from response 12b, a polarity of the response 12b of the person 10 to the second stimulus. The step 87 compares the two polarities to determine whether the first polarity or second polarity has a greater value. The step 88 determines whether person 10 can be rehabilitated.

In accordance with FIG. 14, a first stimulus and a second stimulus are factors capable of eliciting responses from a person 10, pre-determined to be a Luciferian. The first stimulus may be a visual stimulus 14, an audio stimulus 16, a tactile stimulus 18, a multimedia stimulus 19, any other factor or combination of factors capable of eliciting a response from the person 10. The first stimulus may be obvious or unobvious as a test element to determine whether person 10 may be rehabilitated. The second stimulus may be a visual stimulus 14, an audio stimulus 16, a tactile stimulus 18, a multimedia stimulus 19, any other factor or combination of factors capable of eliciting a response from the person 10. The second stimulus may be obvious or unobvious as a test element to determine whether person 10 may be rehabilitated. The person 10, pre-determined to be a Luciferian, may be in a variably controllable environment 23 while subjected to the first stimulus. The person 10, pre-determined to be a Luciferian, may be in a variably controllable environment 23 while subjected to the second stimulus.

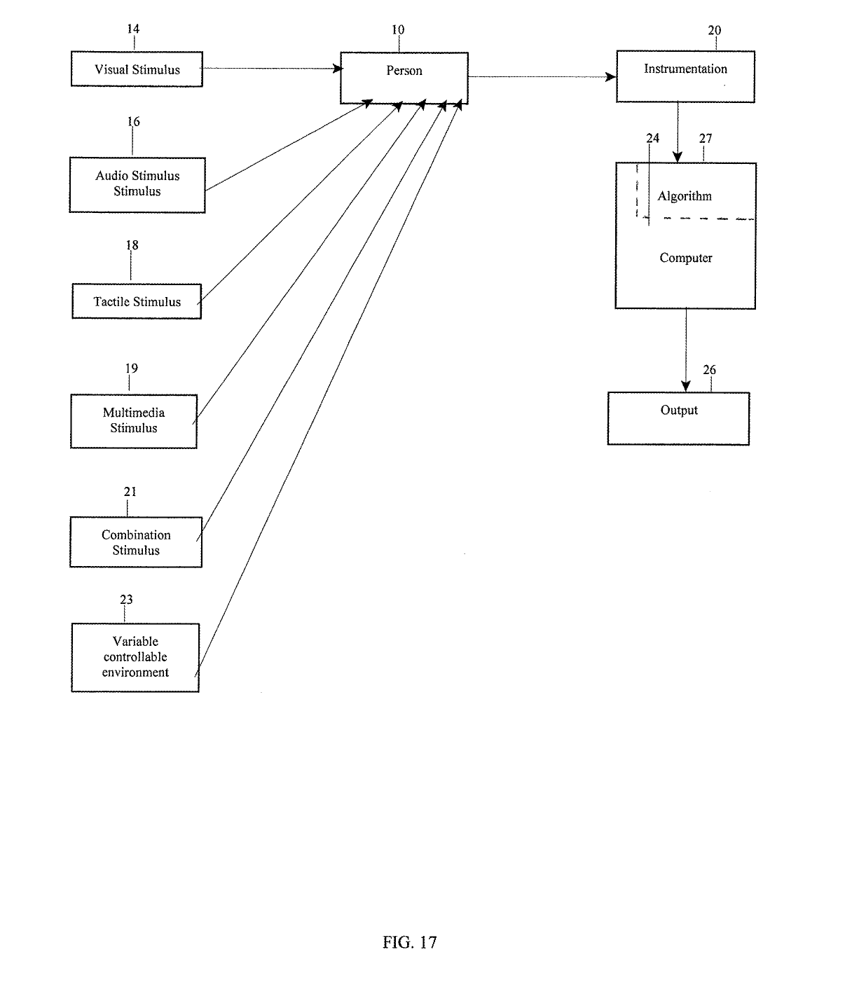

FIG. 14 depicts a recording and processing of responses of a person 10 to a first stimulus and second stimulus to determine whether person 10 (pre-determined to be a Luciferian) may be rehabilitated. A test of FIG. 14 comprises subjecting the person 10, pre-determined to be a Luciferian, to a first stimulus and recording of a first response 12a to the stimulus by a medical instrumentation 20. In accordance with FIG. 17, an algorithm 27 is stored in data processing device 24. The test of FIG. 17 comprises processing response 12a. An algorithm 27 processes the response 12a by determining the first polarity of the response 12a of the person 10 to the first stimulus. The test of FIG. 14 comprises subjecting the person 10, determined to be a Luciferian, to the second stimulus. A response 12b to the second stimulus may be recorded by instrumentation 20. Algorithm 27 processes the response 12b by determining the polarity of the response 12b of the person 10 to the second stimulus. Then, the first polarity and second polarity are compared to determine whether the first polarity or the second polarity has a greater value using algorithm 27. The test of FIG. 17 comprises displaying as output 26 whether a person 10, determined to be a Luciferian, may be rehabilitated. The output 26 may include an expression of whether or not the person 10 may be rehabilitated.

A determination of whether a person 10 (pre-determined to be a Luciferian) may be rehabilitated may be made by a human being rather than by the algorithm 23 within the computer 24, wherein the human being would utilize the same methodology as utilized by algorithm 23.

The first stimulus may be a constructive stimulus or a destructive stimulus. The second stimulus may be a constructive stimulus or a destructive stimulus. An example of constructive stimulus may include a movie clip of a person rescuing animal from a fire. An example may be a picture of a person feeding the poor. Another example of a constructive stimulus may be a movie clip of a person assisting a falling person. An example could be a movie clip showing a criminal's confession to a crime for which another has wrongfully been convicted. A constructive stimulus could be a movie clip of community clean-up efforts. A constructive stimulus may be a movie clip of a person, performing a musical composition. A constructive stimulus may be a movie clip of a person showing respect for a clergy member. A constructive stimulus may be a movie clip of a person working in a flower garden. A constructive stimulus may be a tactile stimulus such as the smell of fresh flowers. A constructive stimulus may be an open page of a magazine showing a successful charity auction. A constructive stimulus may be a newspaper clipping hanging on a wall that praises an employee's successful performance.

An example of a destructive stimulus may include a movie clip or a picture that shows a person hiding in a house and waiting for a victim to arrive. Another example of a destructive stimulus may be a movie clip or picture that shows a person hiding under another's bed. An example of a destructive stimulus may be a movie clip or a picture that shows a person, breaking a bathroom door with an occupied victim inside the bathroom. An example of destructive stimuli may include a movie clip or a picture of a person breaking into a home, entering into the home and murdering a victim. An example of a destructive stimulus may be a movie clip showing a person buried alive. Other examples of destructive stimuli may be a picture of dead animal, a picture of mutilated body of a female, a picture of human torso, a picture of a slit throat, a picture of a dissected body, a movie clip of a person setting a fire, a picture of dungeon or basement, a movie clip of a body in a duffle bag, a picture of a chemical used to degrade a body, a picture of a poisonous substance, a movie clip of a person using a substance to render another unconscious, a movie clip of a child being brutally beaten, a movie clip of cooked human flesh, a movie clip of a person consuming another's blood or body parts, a movie clip of blood under a floor board, and a movie clip of blood seeping through carpet.

A destructive stimulus may be an audio stimulus such as the sound of gun fire. A destructive stimulus may be a person destroying another's sand castle. A destructive stimulus may be a rotten smell. A destructive stimulus may be a movie clip of a person bragging about the murder of a person. A destructive stimulus may be a movie clip showing a person, who appears outwardly positive toward a victim but inwardly is negative towards such individual and the person is open to using any means to destroy the individual, including physically injuring the individual or damaging the individual's property. A destructive stimulus may be a movie clip, showing a person who lies and deceives an individual merely because the person hates the individual. A destructive stimulus may be a scene through a window of a person intentionally stepping on papers dropped by another. A destructive stimulus may be a scene in a doorway where a person smiles at an individual and then frowns at the individual as the individual departs from the presence of the person. A destructive stimulus may be a movie clip of a murderer returning to the scene of a murder to relive the murder experience.

An example of an unobvious stimulus may be an open page of a magazine showing a successful charity auction or a newspaper clipping hanging on a wall that praises an employee's successful performance. An example of an unobvious stimulus may be a scene in a doorway where a person smiles at an individual and then frowns at the individual as the individual departs from the presence of the person. An example of an unobvious stimulus may be a scene through a window of a person intentionally stepping on papers dropped by another.

FIG. 17 shows algorithm 27, instrumentation 20, computer 24 and output 26. Algorithm 27 may record physiological responses 12a and 12b. Physiological response 12a may be an involuntary response or an unintentional response, under the assumption that physiological response 12a could be used to infer the response of the person 10 to the first stimulus. Similarly, physiological response 12b may be an involuntary response or an unintentional response, under the assumption that physiological response 12b could be used to infer the response of the person 10 to the second stimulus.

Algorithm 27 may determine the polarities of the person 10 to the first stimulus and the first stimulus of the responses 12a and 12b. The polarity of the response 12a and the polarity of response 12b indicate whether the response 12a and response 12b are positive or negative. For example, where the first polarity P>0, then the response 12a is positive. If the first polarity P<0, then the response 12a is negative. If the first stimulus S is a destructive stimulus and the first polarity P of response 12a is positive, the Luficerian gains pleasure from the destructive stimulus. If the first stimulus S is a destructive stimulus and the first polarity P of response 12a is negative, the Luficerian does not gain pleasure from the destructive stimulus. If the first stimulus S is a constructive stimulus and the first polarity P of response 12a is positive, the Luficerian gains pleasure from the constructive stimulus. If the first stimulus S is a constructive stimulus and the first polarity P of response 12a is negative, the Luficerian does not gain pleasure from the constructive stimulus.

Algorithm 27 determines whether a Luciferian may be rehabilitated. The output 26 indicates whether a Luciferian may be rehabilitated. If the Luciferian is stimulated with a plurality of constructive stimuli, and then a plurality of destructive stimuli, and the polarity for the plurality of constructive stimuli is greater than the polarity for the plurality of destructive stimuli, then algorithm 27 will express the result that the Luciferian may be rehabilitated. If the Luciferian is stimulated with a plurality of constructive stimuli, and then a plurality of destructive stimuli, and the polarity for the plurality of constructive stimuli is less than the polarity for the plurality of destructive stimuli, the algorithm 27 will express the result that it is unlikely the Luciferian may be rehabilitated.

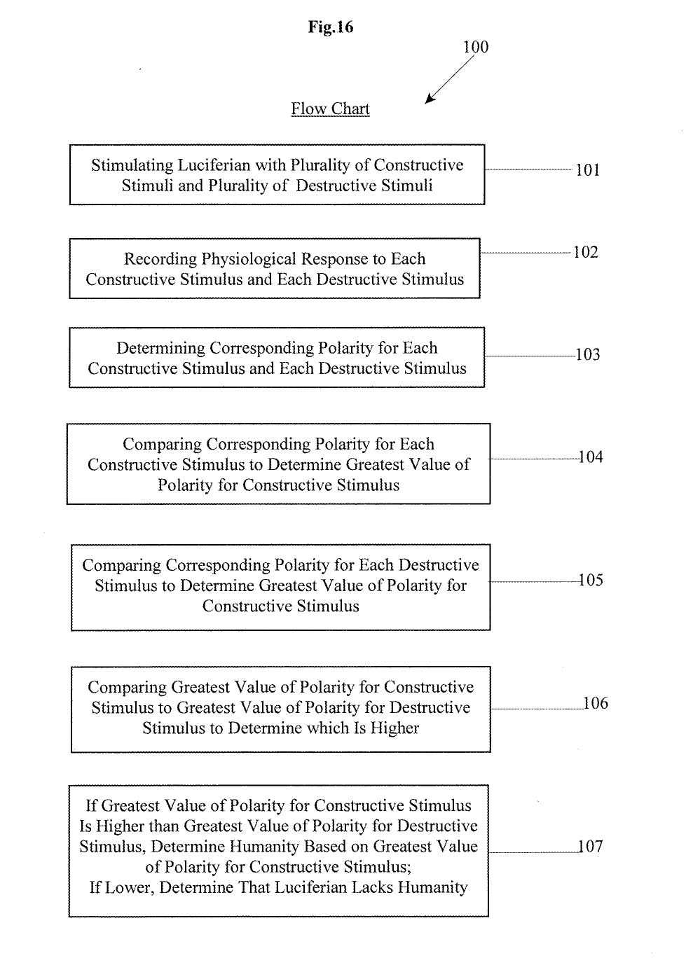

FIG. 15 depicts a flow chart 90 for determining a humanity of a Luciferian. In step 91, a person 10 is stimulated with a plurality of constructive stimuli (S₁, S₂, . . . , S_{N}). In step 92, a physiological response to each constructive stimulus is recorded by the instrumentation 20. In step 93, the algorithm 27 determines a corresponding polarity (P₁, P₂, . . . , P_{N}) for each constructive stimulus. Then, the algorithm 27 compares the polarity of each constructive stimulus to determine the greatest value of polarity. The algorithm 27 determines a humanity of the person 10 based on the constructive stimulus that yields the greatest value of polarity P.

In another aspect, FIG. 16 depicts a flow chart 100 for an alternative method of determining a humanity of a Luciferian. In step 101, a person 10 is stimulated with a plurality of constructive stimuli and a plurality of destructive stimuli. In step 102, a physiological response to each stimulus is recorded by the instrumentation 20. In step 103, the algorithm 27 determines a corresponding polarity for each constructive stimulus and each destructive stimulus. In step 104, the algorithm 27 compares the polarity of each constructive stimulus to determine the greatest value of polarity for constructive stimuli. In step 105, the algorithm 27 compares the polarity of each destructive stimulus to determine the greatest value of polarity for a destructive stimulus. In step 106, the algorithm 27 compares the greatest value of polarity for the constructive stimulus to the greatest value of polarity for the destructive stimulus. In step 106, the algorithm 27 determines whether the greatest value of polarity for the constructive stimulus is higher than the greatest value of polarity for the destructive stimulus. According to step 107, if the greatest value of polarity for the constructive stimulus is higher the greatest value of polarity for the destructive stimulus, the algorithm 27 determines the humanity of the person 10 based on the constructive stimulus that yields the greatest value of constructive stimulus. The algorithm 27 determines at least one humanity and the output 26 expresses the humanity. Thus, the algorithm 27 determines what gives the person 10 more pleasure than destruction. After the humanity of the person 10 is determined, the humanity may be used to counsel the person 10.

Alternatively, the algorithm 27 may determine whether any values of polarities for constructive stimulus are higher than the greatest value of polarity for the destructive stimulus. The algorithm 27 may determine humanities of the person 10 based on the constructive stimuli that yield polarity values higher than the greatest value of polarity for the destructive stimulus.

While particular embodiments of the present invention have been described herein for purposes of illustration, many modifications and changes will become apparent to those skilled in the art. Accordingly, the appended claims are intended to encompass all such modifications and changes as fall within the true spirit and scope of this invention. All embodiments heretofore described are predicted test results and prophetic examples and have not actually been conducted.

What is claimed is:

1. A method for determining whether a Luciferian can be rehabilitated, said method comprising:
stimulating said Luciferian with a first stimulus;
recording a first physiological response by said Luciferian to said first stimulus;
determining, from said first physiological response, a first polarity of said Luciferian's physiological response to said first stimulus;
stimulating said Luciferian with a second stimulus;
recording a second physiological response by said Luciferian to said second stimulus;
determining, from said second physiological response, a second polarity of said Luciferian's physiological response to said second stimulus;
comparing said first polarity to said second polarity to determine whether said second polarity has a greater value than said first polarity; and
determining, from said comparing of said first polarity and said second polarity, whether said Luciferian can be rehabilitated.

2. The method of claim 1, wherein at least one stimulus consists of one stimulus component, wherein the Luciferian's corresponding physiological response consists of one response component.

3. The method of claim 1, wherein said at least one stimulus comprises an N-component stimulus vector {S₁, S₂, . . . , S_{N}} such that N is at least 2, wherein the polarity of the Luciferian's corresponding physiological response to said at least one stimulus is an N-component polarity vector {P₁, P₂, . . . , P_{N}} such that P₁, P₂, . . . , P_{N} respectively corresponds to S₁, S₂, . . . , S_{N}.

4. The method of claim 3, wherein P₁, P₂, . . . , P_{N} are mutually independent.

5. The method of claim 3, wherein one of P₁, P₂, . . . , P_{N} has a dependence on another of P₁, P₂, . . . , P_{N}.

6. The method of claim 1, further comprising: representing a space in a first electronic dataset format;

7. The method of claim 1, further comprising: representing a subspace in a second electronic dataset format.

8. The method of claim 1 wherein said first stimulus is obvious or unobvious.

9. The method of claim 1 wherein said second stimulus is obvious or unobvious.

10. The method of claim 1 wherein at least one physiological response to takes place in a variably controllable environment.

11. The method of claim 1, wherein at least one stimulus comprises an event stream.

12. The method of claim 11, wherein the event stream is in a real-time mode.

13. The method of claim 11, wherein the event stream is in a playback mode.

14. The method of claim 11, wherein the event stream is selected from the group consisting of a movie, a play, a television performance, a sports event, a military event, and combinations thereof.

15. The method of claim 1, wherein at least one stimulus comprises a visual stimulus.

16. The method of claim 1, wherein at least one stimulus comprises an audio stimulus.

17. The process of claim 1, wherein at least one stimulus comprises a tactile stimulus.

18. The method of claim 1, wherein at least one stimulus comprises a multimedia stimulus.

19. The method of claim 1, wherein at least one stimulus comprises a combination stimulus.

20. A method for determining a humanity of a Luciferian, comprising:
stimulating said Luciferian with a plurality of constructive stimuli;
recording a physiological response by said Luciferian corresponding to each constructive stimulus of said plurality of constructive stimuli;
determining, from each constructive stimulus, a corresponding polarity of the Luciferian's response for each constructive stimulus to determine a greatest value; and
determining, from said greatest value, a humanity of said Luciferian.

21. A method for determining a humanity of a Luciferian, comprising:
stimulating said Luciferian with a plurality of destructive stimuli;
recording a physiological response by said Luciferian corresponding to each destructive stimulus of said plurality of destructive stimuli;
determining, from each destructive stimulus, a corresponding polarity of the Luciferian's response for each destructive stimulus to determine a least value; and
determining, from said least value, a humanity of said Luciferian.
